(19) United States
(12) Patent Application Publication
(10) Pub. No.: US 2022/0281956 A1
(43) Pub. Date: Sep. 8, 2022

(54) METHOD FOR PRODUCING A MATERIAL USING COLLAGEN FROM CULTURED ANIMAL CELLS

(71) Applicant: Jellatech, Inc., Raleigh, NC (US)

(72) Inventors: Stephanie Michelsen, Raleigh, NC (US); Juan Camacho, Durham, NC (US); Rob Schutte, Cary, NC (US); Christopher Gilchrist, Durham, NC (US); Megan Koufas Hales, Selma, NC (US)

(73) Assignee: Jellatech, Inc., Raleigh, NC (US)

(21) Appl. No.: 17/686,414

(22) Filed: Mar. 4, 2022

Related U.S. Application Data
(60) Provisional application No. 63/156,636, filed on Mar. 4, 2021.

(51) Int. Cl. C07K 14/78; C12P 21/02

(52) U.S. Cl. CPC C07K 14/78; C12P 21/02

(57) ABSTRACT

Methods to produce a material or a woven or non-woven fabric using collagen isolated from cultured animal cells and/or tissue cultures are described herein. The methods include molding the isolated collagen, treating native collagen and/or an extracellular matrix, and/or combining isolated layered collagen, an extracellular matrix, and/or a connective tissue with other organic and/or non-organic components.

### CROSS-REFERENCE TO RELATED APPLICATIONS SECTION

This application is a U.S. Non-Provisional Patent Application that claims priority to U.S. Provisional Patent Application Ser. 63/156,636 filed on Mar. 4, 2021, the entire contents of which are hereby incorporated for reference in their entirety.

### FIELD OF THE EMBODIMENTS

The field of the invention and its embodiments relate to methods to produce a material or a woven or non-woven fabric using collagen isolated from cultured animal cells and/or tissue cultures.

### BACKGROUND OF THE EMBODIMENTS

Collagen is one of the most abundant components of animal tissues. Collagen products find numerous medicinal and bioengineering uses. For example, collagen is used for wound dressings, as matrices for tissue growth, and as biomaterials for cosmetic surgery, reconstructive surgery, drug delivery system, and scientific research. Most of the collagen products used in these fields are derived from bovine or porcine tissue. Additionally, many procedures describing extraction of collagen from vertebrates, such as cattle, pigs, horses, sheep, poultry, whales, sharks, fish are known. However, such methods harm the animal. As such, improved humane methods to: produce collagen and/or gelatin in animal cell lines and/or tissue explants, isolate and/or extract collagen and/or gelatin from said animal cell lines and/or tissue explants, and produce a material or a woven or non-woven fabric using collagen isolated from cultured animal cells and/or tissue cultures are needed.

### Examples of Related Art Include:

JP2010018575A describes use of jellyfish by a jellyfish extract fraction having cell adhesion inhibitory activity.

JP2004099513A describes a method and a system for extracting and recovering collagen of higher added value through more efficiently treating jellyfish.

JP3696018B2 describes a crude extraction process for useful substances (such as collagen) from jellyfish that includes: crushing jellyfish, shredding the jellyfish into pieces, decomposing the jellyfish, solubilizing the jellyfish, and purifying the jellyfish.

JP2007051191A describes a method for recovering collagen that includes the steps of: freezing jellyfish, thawing the frozen jellyfish to activate an endogenous enzyme of jellyfish to start the decomposition reaction of jellyfish, mixing the thawed jellyfish to solubilize the collagen of jellyfish in a native state to form a neutral salt solution containing native collagen, and recovering the native collagen from the neutral salt solution.

JP2008031106A describes a method that comprises a low-temperature storage step for storing jellyfish at a low temperature for activating an endogenous enzyme of jellyfish to cause it to initiate a decomposition reaction of the jellyfish and solubilizing collagen of the jellyfish in an unmodified state to form a neutral salt solution containing unmodified collagen. The method also includes a recovery step for recovering the unmodified collagen from the neutral salt solution.

WO2014157854A1 and U.S. Published Patent Application No. 2016/0052962 A1 describe a method for isolating collagen from jellyfish through use of radiation.

WO2015005830A1 describes a method for producing collagen from jellyfish.

WO2015012682A2 describes an improved process for extracting collagen from aquatic animals (such as jellyfish), comprising alkaline treatment, followed by acidic treatment in combination with an orderly sequence of physical and/or mechanical treatments and precipitation of collagen using a salt solution. The process increases the yield and quality of the collagen while decreasing the production time and is more cost-effective than the processes known heretofore.

WO2018220396A1 describes hydrolyzed collagen types I, II, and V powder compositions, method of preparing the compositions, and use of the compositions in treating a variety of ailments. The collagen is derived from an organism, such as: jellyfish, anemone, echinoderms, limpets, mussels, sea cucumbers, bovine, porcine, rodent, equine or finfish. The jellyfish may be selected from the list consisting of Rhizostomas pulmo, Rhopilema esculentum, Rhopilema nomadica, Stomolophus meleagris, Aurelia sp., Nemopilema nomurai or a combination thereof.

Some systems exist to extract collagen and/or gelatin from an animal, such as a jellyfish. However, such systems harm the animal. Moreover, the means of operation of such systems are substantially different from the present disclosure, as the other inventions fail to solve all the problems taught by the present disclosure.

### SUMMARY OF THE EMBODIMENTS

The present invention and its embodiments relate to methods to produce a material or a woven or non-woven fabric using collagen isolated from cultured animal cells and/or tissue cultures.

A first embodiment of the present invention describes a method. The method includes numerous process steps, such as: utilizing a media to cultivate a (continuous) cell line of an animal. The animal may be an invertebrate animal or a vertebrate animal. In other examples, the animal may be: a marine animal, a porcine animal, a bovine animal, or an avian animal. The method further includes extracting collagen from the (continuous) cell line of the animal through use of a material, extraction using an external field (such as ultrasonication) and an automatized method (such as chromatography), and/or a first process. The material may be a buffer, an enzyme, an acid, and/or a base, among others. The enzyme may be collagenase or pepsin. Further, the first process may include lyophilizing and/or spray drying.

The method may further include utilizing the extracted collagen to produce a product through a second process. The product may be a woven or non-woven fabric or another material. The second process may include molding the extracted collagen to take on and hold a shape via a treatment. The treatment may be: a treatment with an acid, a treatment with a base, a treatment with one or more temperature changes, a treatment with an enzyme, a treatment with a buffer, a treatment with a solvent, and/or a mechanical treatment, among others.

A second embodiment of the present invention describes a method to create a material from native collagen, an extracellular matrix and/or a connective tissue produced by animal cells. The method includes numerous process steps, such as: decellularizing adherent cell cultures through a first process, layering adherent cell cultures on top of one another through a second process, isolating a resulting layer through a third process, and using the resulting layer to create the material through a fourth process. Each of the first, third, and fourth processes include: a treatment with an acid, a treatment with a base, a treatment with one or more temperature changes, a treatment with an enzyme, a treatment with a buffer, a treatment with a solvent, and/or a mechanical treatment, among other treatments. Moreover, the second process includes: use of consecutive cultures where a previous culture is decellularized prior to a following culture, use of the consecutive cultures where a previous culture is not decellularized prior to the following culture, and/or use of the consecutive cultures that are decellularized at any time.

A third embodiment of the present invention describes a method to create a composite material. The method includes numerous process steps, such as: combining isolated (layered) collagen, an extracellular matrix and/or a connective tissue produced by animal cells with one or more organic and/or non-organic components. The organic component may include: a plant-derived component, an animal cell culture-derived component, and/or a fungus-derived component, among others. The non-organic component may include: a chemical component, a polymer component, a natural component, and/or a synthetic component, among others.

In general, the present invention succeeds in conferring the following benefits and objectives.

It is an objective of the present invention to provide a humane method to extract collagen from the (continuous) animal cell line that does not harm the animal.

It is an objective of the present invention to provide a method to utilize extracted collagen to produce a product.

It is an objective of the present invention to provide a method to create a material from native collagen, an extracellular matrix, and/or a connective tissue produced by animal cells.

It is an objective of the present invention to create a composite material by combining isolated (layered) collagen, an extracellular matrix, and/or a connective tissue produced by animal cells with one or more organic and/or non-organic components.

### BRIEF DESCRIPTION OF THE DRAWINGS

FIG. 1 depicts a block diagram of a first method, according to at least some embodiments disclosed herein.

FIG. 2 depicts a block diagram of a second method, according to at least some embodiments disclosed herein.

FIG. 3 depicts a block diagram of a third method, according to at least some embodiments disclosed herein.

### DESCRIPTION OF THE PREFERRED EMBODIMENTS

The preferred embodiments of the present invention will now be described with reference to the drawings. Identical elements in the various figures are identified with the same reference numerals. Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

### Collagen

Collagen is the predominant structural protein in the extracellular matrix of connective tissues in animals and is widely used in tissue regeneration and other industrial applications. See, K. E. Kadler, et al., “Collagens at a Glance,” Journal of Cell Science, 2007, 120, Pages 1955-1958, the entire contents of which are hereby incorporated by reference in their entirety. Collagen may be fibrillar or non-fibrillar. Fibrillar collagen includes Type I, Type II, Type III, Type V, and Type XI. Non-fibrillar collagen includes fibril associated collagens with interrupted triple helices (or FACIT) (e.g., Type IX, Type XII, Type XIV, Type XIX, and Type XXI), short chain collagen (Type VIII and Type X), basement membrane collagen (e.g., Type IV), multiple triple helix domains with interruptions (or Multiplexin) (e.g., Type XV and Type XVIII), membrane associated collagens with interrupted triple helices (or MACIT) (e.g., Type XIII and Type XVII), and others (e.g., Type VI and Type VII). The five most common types of collagen include Type I (e.g., the main component of the organic part of bone), Type II (e.g., the main collagenous component of cartilage), Type III (e.g., the component of reticular fibers), Type IV forms basal lamina, the epithelium-secreted layer of the basement membrane), and Type V (e.g., cell surfaces, hair, and placenta).

Commercially available collagen-based agents are usually derived from bovine and porcine sources. However, collagen of bovine origin are associated with the transmission of bovine spongiform encephalopathy (BSE) (or mad cow disease) and transmissible spongiform encephalopathy (TSE), as well as potential viral vectors that could be transmissible to humans. See, M. Ogawa, et al., “Biochemical Properties of Bone and Scale Collagens Isolated from the Subtropical Fish Black Drum (Pogonia cromis) and Sheepshead Seabream (Archosargus probatocephalus),” Food Chem., 2004, 88(4), Pages 495-501; H. Li, et al., “Studies on Bullfrog Skin Collagen,” Food Chem., 2004, 84(1), Pages 65-9; and J. P. Widdowson J. P., et al., “In Vivo Comparison of Jellyfish and Bovine Collagen Sponges as Prototype Medical Devices,” J. Biomed. Mater. Res. Part B Appl. Biomater, 2018, 106, Pages 1524-1533, the entire contents of which are hereby incorporated by reference in their entirety. Moreover, porcine collagen can also cause religious and/or ethical problems. See, B. Hoyer, et al., “Jellyfish Collagen Scaffolds for Cartilage Tissue Engineering,” Acta Biomater, 2014, 10, Pages 883-892, the entire contents of which are hereby incorporated by reference in their entirety. Furthermore, there are growing regulatory concerns around the continued use of mammalian collagens, as they are considered a pathological risk for transmitted diseases, such as avian influenza, swine influenza, and tooth-and-mouth disease. See, F. Subhan, et al., “Marine Collagen: an Emerging Player in Biomedical Applications,” J. Food Sci. Technol., 2015, 52, Pages 4703-4707, the entire contents of which are hereby incorporated by reference in their entirety. Some have also shown that different mammalian collagen-based materials induce pro-inflammatory tissue responses due to their purification processes. See, T. Miyata, et al., “Collagen Engineering for Biomaterial Use. Clin. Mater,” 1992, 9, Pages 139-148; and J. M. Aamodt, et al., “Extracellular Matrix-Based Biomaterial Scaffolds and the Host Response,” Biomaterials, 2016, 86, Pages 68-82, the entire contents of which are hereby incorporated by reference in their entirety.

Due to these deficiencies, marine organisms have gained interest as alternative, non-mammalian collagen sources for biomaterial applications because of potential medical and economic advantages. Interestingly, marine organisms present an attractive alternative, due to lack of BSE risk and potential viral vectors. Specifically, jellyfish have been viewed as one such alternative, since jellyfish are rich in minerals, proteins, and collagen. See, Y. P. Hsieh, et al., “Jellyfish as Food,” Hydrobiologia, 2001,451(1-3), Pages 11-7, the entire contents of which are hereby incorporated by reference in their entirety.

The isolation and characterization of collagen from different fish and jellyfish species has been described. See, S. Addad, et al., “Isolation, Characterization and Biological Evaluation of Jellyfish Collagen for Use in Biomedical Applications,” Mar. Drugs, 2011, 9, Pages 967-983; Z. Rastian, et al., “Type I Collagen from Jellyfish Catostylus mosaicus for Biomaterial Applications,” ACS Biomater. Sci. Eng, 2018, 4, Pages 2115-2125; S. Krishnan, et al., “Preparation and Biomedical Characterization of Jellyfish (Chrysaora quinquecirrha) Collagen from Southeast Coast of India,” Int. J. Pharm. Pharm. Sci, 2013, 5, Pages 698-701; and S. Yamada, et al., “Potency of Fish Collagen as a Scaffold for Regenerative Medicine,” Biomed Res. Int, 2014, Page 302932, the entire contents of which are hereby incorporated by reference in their entirety. Moreover, the usability of marine collagens has already been analyzed and it has been shown that jellyfish collagen is non-toxic and induces a higher cell viability of fibroblasts and osteoblasts compared to bovine collagen. See, S. Addad, et al.. Additionally, others have conducted studies that tested different Mediterranean jellyfish species in order to investigate the different methods of collagen purification. See, S. Addad, et al.. Based on this study, it was concluded that the best collagen yield was obtained using Rhizostoma pulmo (R. pulmo), and furthermore, upon biological analysis, the cytotoxicity of R. pulmo collagen was no different in comparison to mammalian collagen. See, S. Addad, et al..

Further studies supporting the potential of jellyfish collagen's biocompatibility have been conducted, which included cytotoxicity tests, measurements of pro-inflammatory cytokine secretion and antibody secretion, as well as the population change of immune cells after in vivo implantation. See, S. Addad, et al.. In one such study, it was found that the number of dendritic cells (CD11c+) and macrophages (F4/80+) were similar in jellyfish collagen implanted into mice as to those of bovine- and gelatin-implanted mice. See, E. Song, et al., “Collagen Scaffolds Derived from a Marine Source and Their Biocompatibility,” Biomaterials, 2006, 27, Pages 2951-2961, the entire contents of which are hereby incorporated by reference in their entirety. Hence, this study concluded that the jellyfish collagen scaffolds were able to induce a comparable immune response to that caused by bovine collagen or gelatin. See, E. Song, et al..

Additionally, another study reported that the peptides derived from R. esculentum could reduce the blood pressure in spontaneously hypertensive rats and be used as antihypertensive compounds in functional foods. See, X. Liu, et al., “Purification and Characterization of Angiotensin I Converting Enzyme Inhibitory Peptides from Jellyfish Rhopilema esculentum,” Food Res Int., 2013, 50(1), Pages 339-43, the entire contents of which are hereby incorporated by reference in their entirety. Another group reported that proteins isolated from jellyfish R. esculentum showed strong antioxidant activity and might be applied in the food and pharmaceutical industries. See, H. Yu, et al., “In vitro Determination of Antioxidant Activity of Proteins from Jellyfish Rhopilema esculentum,” Food Chem, 2006, 95(1), Pages 123-30, the entire contents of which are hereby incorporated by reference in their entirety.

Jellyfish collagen possesses the common feature of collagen molecules exhibiting a triple helix structure and is resistant to pepsin digestion. See, A. Miki, et al., “Structural and Physical Properties of Collagen Extracted from Moon Jellyfish under Neutral pH Conditions,” Biosci Biotechnol Biochem, 2015, 79, Pages 1603-1607; and B. Hoyer, et al., the entire contents of which are hereby incorporated by reference in their entirety. Moreover, jellyfish collagen, which can be defined as “collagen type 0” due to its homogeneity to the mammalian types I, II, III, V, and IX and its batch-to-batch consistent producibility, is of special interest for different medical applications related to (bone) tissue regeneration as an alternative to mammalian collagen-based biomaterials. See, Iris Flaig, et al., “In Vivo Analysis of the Biocompatability and Immune Responses of Jellyfish Collagen Scaffolds and its Suitability for Bone Regeneration,” International Journal of Molecular Sciences, 2020, 21(12), Page 4518, the entire contents of which are hereby incorporated by reference in their entirety.

Some have shown that collagens extracted from several species of jellyfish exhibit unique functional properties. For example, species of Nemopilema are known to be edible and harmless jellyfish, and their collagen stimulates immune reactions through the TLR4 signaling pathway. See, H. Morishige, et al., “Immunostimulatory Effects of Collagen from Jellyfish in vivo,” Cytotechnology, 2011, 63, Pages 481-492; and A. B. Putra, et al., “Jellyfish Collagen Stimulates Production of TNF-α and IL-6 by J774.1 Cells Through Activation of NF-κB and JNK via TLR4 Signaling Pathway,” Mol Immunol, 2014, 58, Pages 32-37, the entire contents of which are hereby incorporated by reference in their entirety. Others have reported that collagen extracted from this species accelerates cartilage differentiation from mesenchymal stem cells. See, B. Hoyer, et al.. Collagen extracted from Aurelia species (moon jellyfish) possesses the unique property of high water solubility that collagens from other species of jellyfish do not. See, A. Miki, et al.. Despite these promising benefits, the structures and functions of collagenous proteins in invertebrates, such as jellyfish, have not been fully understood.

Traditional methods to extract collagen from a collagen-containing matter may include numerous process steps. In examples, the phrase “collagen-containing matter” refers to a source material from which the collagen is to be extracted. In some embodiments, the collagen-containing matter is derived from an organism, such as: a jellyfish, an anemone, an echinoderm, a limpet, a mussel, a sea cucumber, a bovine, a porcine, a rodent, an equine, or a finfish. Such method may be described in WO 2018/220396 A1, the contents of which are hereby incorporated by reference in their entirety. One such illustrative method includes: (a) incubating the collagen-containing matter in an acidic solution for at least 1 hour at a temperature in the range of about 4° C. to about 37° C. to form an incubant; (b) diafiltrating the incubant from step (a) to substantially purify solubilized collagen within the incubant, thereby forming a retentate; (c) separating the soluble and insoluble matter of the retentate obtained from step (b) to remove the remaining insoluble matter; and (d) optionally repeating steps (a) and (b) on the remaining insoluble matter, where the soluble matter obtained from step (c) is a substantially pure collagen solution. However, such methods harm the animal. As such, humane alternatives are needed to extract collagen from organisms.

### Gelatin

Gelatin is typically derived from denatured collagen via acid hydrolysis, alkaline hydrolysis, and enzyme hydrolysis. Type A and Type B gelatin commonly used in food industry are derived by acid and alkaline processes, respectively. Type A gelatin produced from jellyfish can be used as an alternative source of gelatin for food application. See, U. Rodsuwan, et al., “Functional Properties of Type A Gelatin from Jellyfish (Lobonema smithii),” International Food Research Journal, 2016, 23(2), Pages 507-514, the entire contents of which are hereby incorporated by reference in their entirety.

### Methods

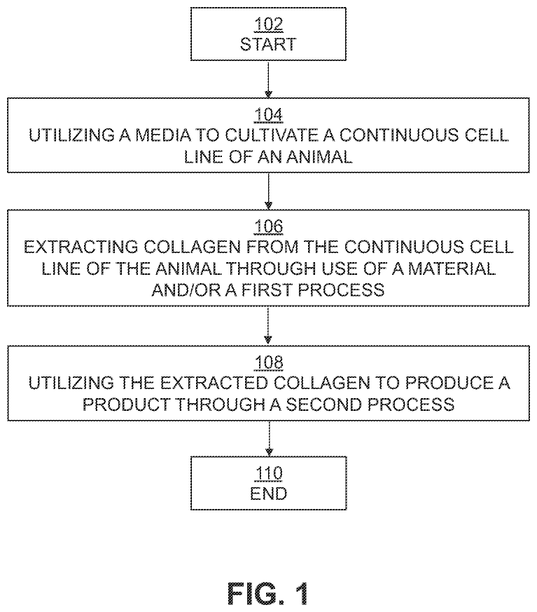

FIG. 1 depicts a first method of the present invention. The first method of FIG. 1 begins with a process step 102. A process step 104 follows the process step 102 and includes utilizing a medium to cultivate a (continuous) cell line of an animal. The animal may be an invertebrate animal or a vertebrate animal. In other examples, the animal may be a marine animal, a porcine animal, a bovine animal, and/or an avian animal, among other examples not explicitly listed herein. In preferable examples, the animal may be a marine animal, and more specifically, the jellyfish.

A process step 106 follows the process step 104 and includes extracting collagen from the (continuous) cell line of the animal through use of a material and/or a first process. The material may be a buffer, an enzyme, an acid, and/or a base. In some examples, the enzyme may include collagenase and/or pepsin, among other examples. The first process may include lyophilizing and/or spray drying. As described herein, “lyophilization” or “freeze-drying” is a low temperature dehydration process that involves freezing the product, lowering pressure, then removing the ice by sublimation. See, P. Fellows, “Freeze drying and freeze concentration,” Food processing technology: Principles and practice, 2017, 4th ed., Kent: Woodhead Publishing/Elsevier Science, Pages 929-940, the entire contents of which are hereby incorporated by reference in their entirety.

It should be appreciated that other methods may be used to extract collagen. For example, methods to extract collagen from a cultured animal explant through use of a material and/or a process are also described herein. In some examples, the animal explant includes a vertebrate animal explant and/or an invertebrate animal explant. In other examples, the animal explant includes an avian animal explant, a bovine animal explant, a porcine animal explant, and/or a marine animal explant, among other examples not explicitly listed herein. The marine animal explant is a jellyfish explant, a jellyfish polyp explant, a jellyfish medusae explant, and/or a marine sponge explant, among other examples not explicitly listed herein.

A process step 108 follows the process step 106 and includes utilizing the extracted collagen to produce a product through a second process. The product includes a woven or non-woven fabric or another material. In a first example, the second process includes molding the extracted collagen to take on and hold a shape via a treatment. The treatment may include: treatment with an acid, a treatment with a base, a treatment with one or more temperature changes, a treatment with an enzyme, a treatment with a buffer, a treatment with a solvent, and/or a mechanical treatment, among other examples not explicitly listed herein. A process step 110 follows the process step 108 and concludes the method of FIG. 1.

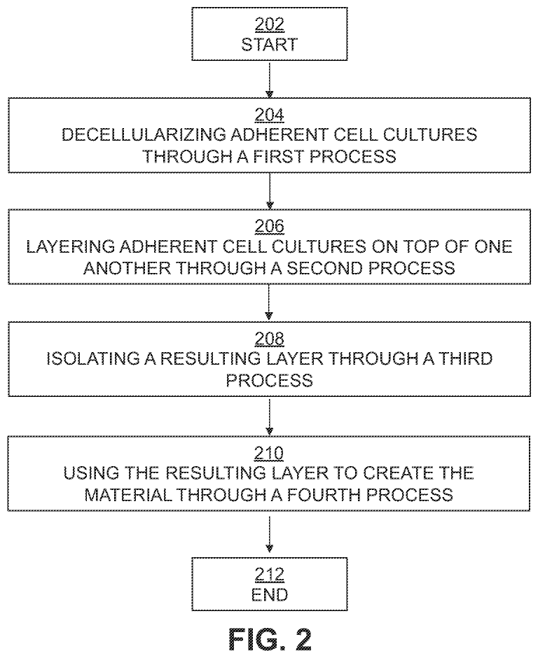

FIG. 2 depicts a second method of the present invention. The second method of FIG. 2 includes numerous process steps for creating a material from native collagen, an extracellular matrix and/or a connective tissue produced by animal cells. The second method of FIG. 2 begins at a process step 202, which is followed by a process step 204 that includes decellularizing adherent cell cultures through a first process. It should be appreciated that, as described herein, “adherent cells” are cells that must be attached to a surface to grow. The first process may include a treatment with an acid, a treatment with a base, a treatment with one or more temperature changes, a treatment with an enzyme, a treatment with a buffer, a treatment with a solvent, and/or a mechanical treatment, among other treatments.

A process step 206 follows the process step 204 and includes layering adherent cell cultures on top of one another through a second process. The second process of the process step 206 includes: use of consecutive cultures where a previous culture is decellularized prior to a following culture, use of the consecutive cultures where a previous culture is not decellularized prior to the following culture, and/or use of the consecutive cultures that are decellularized at any time.

A process step 208 follows the process step 206 and includes: isolating a resulting layer through a third process. The third process may include a treatment with an acid, a treatment with a base, a treatment with one or more temperature changes, a treatment with an enzyme, a treatment with a buffer, a treatment with a solvent, and/or a mechanical treatment, among other treatments.

A process step 210 follows the process step 208 and includes: using the resulting layer to create the material through a fourth process. The fourth process may include a treatment with an acid, a treatment with a base, a treatment with one or more temperature changes, a treatment with an enzyme, a treatment with a buffer, a treatment with a solvent, and/or a mechanical treatment, among other treatments. A process step 212 follows the process step 210 to conclude the method of FIG. 2.

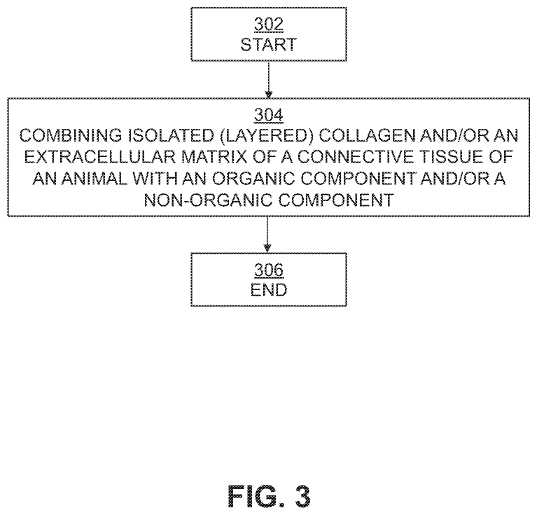

FIG. 3 depicts a third method of the present invention. The third method of FIG. 3 includes numerous process steps for creating a composite material. The third method of FIG. 3 begins at a process step 302, which is followed by a process step 304 that includes combining isolated (layered) collagen, an extracellular matrix and/or a connective tissue produced by animal cells with one or more organic and/or a non-organic components. The organic component may be a plant-derived component, an animal cell culture-derived component, and/or a fungus-derived component, among others not explicitly listed herein. The non-organic component may include: a chemical component, a polymer component, a natural component, and/or a synthetic component, among other components not explicitly listed herein. A process step 306 follows the process step 304 to conclude the third method of FIG. 3.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others or ordinary skill in the art to understand the embodiments disclosed herein.

When introducing elements of the present disclosure or the embodiments thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A method comprising:
utilizing a media to cultivate a (continuous) cell line of an animal;
extracting collagen from the (continuous) cell line of the animal through use of a material and/or a first process; and
utilizing the extracted collagen to produce a product through a second process.

2. The method of claim 1, wherein the media is selected from the group consisting of: Minimum Essential Medium (MEM), Dulbecco's Modified Eagle Medium (DMEM)-High glucose, DMEM-F12, FM, DMEM advanced, and RPMI 1640 advanced.

3. The method of claim 1, wherein the animal is selected from the group consisting of: an invertebrate animal and a vertebrate animal.

4. The method of claim 1, wherein the animal is selected from the group consisting of: a marine animal, a porcine animal, a bovine animal, and an avian animal.

5. The method of claim 1, wherein the material is selected from the group consisting of: a buffer, an enzyme, an acid, and a base.

6. The method of claim 5, wherein the enzyme is selected from the group consisting of: collagenase and pepsin.

7. The method of claim 1, wherein the first process comprises lyophilizing.

8. The method of claim 1, wherein the product comprises a woven fabric, a non-woven fabric, or another material.

9. The method of claim 1, wherein the second process comprises molding the extracted collagen to take on and hold a shape via a treatment.

10. The method of claim 9, wherein the treatment is selected from the group consisting of: a treatment with an acid, a treatment with a base, a treatment with one or more temperature changes, a treatment with an enzyme, a treatment with a buffer, a treatment with a solvent, and a mechanical treatment.

11. A method to create a material from native collagen, an extracellular matrix, and/or a connective tissue produced by animal cells, the method comprising:
decellularizing adherent cell cultures through a first process;
layering adherent cell cultures on top of one another through a second process;
isolating a resulting layer through a third process; and
using the resulting layer to create the material through a fourth process.

12. The method of claim 11, wherein the first process is selected from the group consisting of: a treatment with an acid, a treatment with a base, a treatment with one or more temperature changes, a treatment with an enzyme, a treatment with a buffer, a treatment with a solvent, and a mechanical treatment.

13. The method of claim 11, wherein the second process is selected from the group consisting of: use of consecutive cultures where a previous culture is decellularized prior to a following culture, use of the consecutive cultures where a previous culture is not decellularized prior to the following culture, and use of the consecutive cultures that are decellularized at any time.

14. The method of claim 11, wherein the third process is selected from the group consisting of: a treatment with an acid, a treatment with a base, a treatment with one or more temperature changes, a treatment with an enzyme, a treatment with a buffer, a treatment with a solvent, and a mechanical treatment.

15. The method of claim 11, wherein the fourth process is selected from the group consisting of: a treatment with an acid, a treatment with a base, a treatment with one or more temperature changes, a treatment with an enzyme, a treatment with a buffer, a treatment with a solvent, a treatment with lyophilization, a treatment with irradiation, a thermal treatment, and a mechanical treatment.

16. A method to create a composite material, the method comprising:
combining isolated (layered) collagen, an extracellular matrix, and/or a connective tissue produced by animal cells with an organic component and/or a non-organic component.

17. The method of claim 16, wherein the organic component is selected from the group consisting of: a plant-derived component, an animal cell culture-derived component, and a fungus-derived component.

18. The method of claim 16, wherein the non-organic component is selected from the group consisting of: a chemical component, a polymer component, a natural component, and a synthetic component.
